(19) United States
(12) Patent Application Publication
(10) Pub. No.: US 2025/0261933 A1
(43) Pub. Date: Aug. 21, 2025

(54) Methods and Systems to Identify Actual Esophageal Tissue Changes During Cardiac Ablation

(71) Applicant: REGENTS OF THE UNIVERSITY OF MICHIGAN, Ann Arbor, MI (US)

(72) Inventors: Grant Kruger, Ann Arbor, MI (US); Hakan Oral, Ann Arbor, MI (US)

(21) Appl. No.: 18/857,653

(22) PCT Filed: Apr. 17, 2023
(86) PCT No.: PCT/US23/18785

§ 371 Date: Oct. 17, 2024

Related U.S. Application Data
(60) Provisional application No. 63/331,914, filed on Apr. 18, 2022.

(51) Int. Cl. A61B 17/02; A61B 17/00; A61B 18/00; A61B 18/14

(52) U.S. Cl. CPC A61B 17/0218; A61B 18/1492; A61B 2017/00057; A61B 2017/00119; A61B 2017/00566; A61B 2018/00351; A61B 2018/00577; A61B 2018/00678; A61B 2018/00773; A61B 2018/00797; A61B 2018/00892; A61B 2018/00898

(57) ABSTRACT

Methods and systems to identify actual esophageal tissue changes (e.g., during cardiac ablation) are disclosed. An example system includes a probe and a data analysis unit. The probe is configured for positioning in a patient's esophagus and includes a sheath, and a plurality of tendrils extendible by a user from an end of the sheath within the esophagus. Each tendril has a plurality of nodes spaced apart along the tendril, wherein each node of the plurality of nodes is configured to be used to introduce signals into esophageal tissue and/or receive signals affected by esophageal tissue. The data analysis unit is configured to determine a value of a characteristic indicative of actual change to esophageal tissue between two nodes based on a signal received using at least one of the nodes, and to provide an indication of actual esophageal tissue change based on the determined value.

### FIELD OF THE DISCLOSURE

This disclosure relates generally to cardiac ablation, and, more particularly, to methods and systems to identify actual esophageal tissue changes during cardiac ablation.

### BACKGROUND

During a cardiac ablation procedure, tissue of the heart wall is intentionally damaged to limit the propagation of electrical impulses. For a number of reasons, tissue change and/or damage can extend beyond the heart. For example, heat-induced damage to the esophagus may occur, which, while rare, represents a dangerous medical condition for the patient. Esophageal monitoring is traditionally used to detect signs that esophageal tissue change and/or damage may be occurring so that the ablation process can be terminated before excessive change and/or damage results. Traditionally, esophageal probes with a single or multiple temperature sensors have been employed for this purpose. However, the large area of the esophageal wall that must be interrogated, limited thermal spread from the procedure site, and/or limited sensor sensitivity can all limit the effectiveness of existing methods. Furthermore, increased esophageal tissue temperature is only a secondary marker or indication of potential esophageal tissue damage (i.e., an indicator that esophageal tissue change and/or damage may be imminent). Existing methods are not able to determine whether or the extent to which the esophageal tissue has been actually changed and/or damaged by any increases in temperature. Thus, using temperature sensors, actual esophageal tissue change and/or damage may be missed, which potentially results in more severe change and/or damage to the esophagus, and/or a cardiac ablation procedure may be prematurely halted, which potentially results in a remaining, at least partially-untreated heart condition. Thus, there is a need for methods and systems to identify actual esophageal tissue changes and/or damage during cardiac ablation.

### SUMMARY

In an embodiment, a system for detecting changes in esophageal tissue properties includes: a probe configured for positioning in a patient's esophagus, the probe including: one or more tendrils that can be manipulated be a user within the esophagus, wherein the one or more tendrils are configured to contact respective esophageal tissue at different locations in the esophagus, wherein each tendril includes a plurality of nodes spaced apart along the tendril, and wherein each node of the plurality of nodes is configured to be used to introduce signals into esophageal tissue and/or receive signals affected by esophageal tissue; and a data analysis unit configured to: introduce a first signal into esophageal tissue using a first node; determine a value of a characteristic indicative of changes to esophageal tissue at the first node or between a second node and a third node on the same tendril or different tendrils; and provide an indication of changing tissue properties from a determined baseline value.

In another embodiment, a method for detecting changes in esophageal tissue properties due to cardiac ablation includes: deploying a probe in a patient's esophagus, wherein the probe includes a sheath and a plurality of tendrils extendible by a user from an end of the sheath, wherein the plurality of tendrils are configured to contact respective esophageal tissue at different locations in the esophagus when extended from the end of the sheath, wherein each tendril of the plurality of tendrils includes a plurality of nodes spaced apart along the tendril, and wherein each node of the plurality of nodes is configured to introduce signals into esophageal tissue and/or receive signals affected by esophageal tissue; introducing a first signal into esophageal tissue using a first node; determining, during a cardiac ablation procedure and based on a second signal received using a second node, a value of a characteristic indicative of actual changes to esophageal tissue properties of the patient's esophagus in proximity to a cardiac ablation site and between the second node and a third node; determining, during the cardiac ablation procedure and based on the value, whether actual esophageal tissue change has occurred; and providing, during the cardiac ablation procedure, an indication of actual esophageal tissue change when determining that actual esophageal tissue change has occurred.

In yet another embodiment, a non-transitory, machine-readable storage medium stores instructions that, when executed, cause a system for detecting actual esophageal tissue changes due to cardiac ablation to, after a probe is deployed in a patient's esophagus, wherein the probe includes a sheath and a plurality of tendrils extendible by a user from an end of the sheath, wherein the plurality of tendrils are configured to contact respective esophageal tissue at different locations in the esophagus when extended from the end of the sheath, wherein each tendril of the plurality of tendrils includes a plurality of nodes spaced apart along the tendril, and wherein each node of the plurality of nodes is configured to introduce signals into esophageal tissue and/or receive signals affected by esophageal tissue: introduce a first signal into esophageal tissue using a first node; determine, during a cardiac ablation procedure and based on a second signal received using a second node, a value of a characteristic indicative of actual change to esophageal tissue of the patient's esophagus in proximity to a cardiac ablation site and between the second node and a third node; determine, during the cardiac ablation procedure and based on the value, whether actual esophageal tissue change has occurred; and provide, during the cardiac ablation procedure, an indication of actual esophageal tissue change when determining that actual esophageal tissue change has occurred.

### BRIEF DESCRIPTION OF THE DRAWINGS

FIG. 1 illustrates an example esophageal tissue damage monitoring system constructed in accordance with disclosed embodiments.

FIG. 2 illustrates an example end of the probe of FIG. 1 having a plurality of extendible and retractable tendrils.

FIGS. 3A, 3B and 3C illustrate an example deployment of the probe of FIGS. 1 and 2.

FIG. 4 is an example cross-section of one of the tendrils of FIGS. 1, 2 and 3A-C.

FIG. 5 is a block diagram illustrating an example data analysis unit that may be used to implement the data analysis unit of FIG. 1.

FIG. 6 illustrates an example bioimpedance measurement technique using the example tendrils of FIGS. 1, 2, 3A-C and 4.

FIGS. 7 and 8 are example visualizations of electrical tissue conductivity.

FIG. 9 illustrates an example shear-wave velocity measurement technique using the example tendrils of FIGS. 1, 2, 3A-C and 4.

FIG. 10 is a flowchart representative of an example process that may be implemented by the data analysis units of FIGS. 1 and 5.

FIG. 11 is a flowchart representative of another example process that may be implemented by the data analysis units of FIGS. 1 and 5.

FIGS. 12A and 12B are diagrams illustrating an example soft robotics actuator and deployment method that may be used to arch a tendril.

FIG. 13 is a diagram illustrating an example shape memory alloy that may be used to arch a tendril.

FIGS. 14A and 14B are diagrams illustrating an example pre-strained tendril and deployment method that may be used to arch a tendril.

FIG. 15 is a block diagram of an example computing system to implement the various user interfaces, methods, functions, etc., for identifying actual esophageal tissue damage during cardiac ablation, in accordance with the disclosed embodiments.

The figures depict embodiments of this disclosure for purposes of illustration only. One skilled in the art will readily recognize from the following discussion that alternate embodiments of the structures and methods illustrated herein may be employed without departing from the principles set forth herein.

In general, the same reference numbers will be used throughout the drawing(s) and accompanying written description to refer to the same or like parts. The figures are not to scale. Connecting lines or connectors shown in the various figures presented are intended to represent example functional relationships and/or physical or logical couplings between the various elements.

### DETAILED DESCRIPTION

Reference will now be made in detail to non-limiting examples, some of which are illustrated in the accompanying drawings.

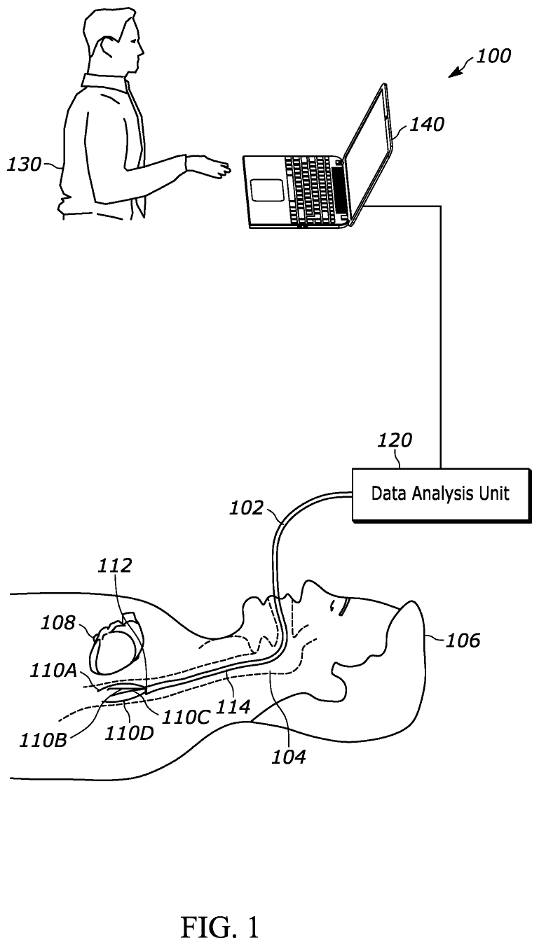

FIG. 1 illustrates an example esophageal tissue damage monitoring system 100 constructed in accordance with disclosed embodiments. The monitoring system 100 includes an example flexible probe 102 that can be positioned within the esophagus 104 of a patient 106 to monitor for actual and/or potential esophageal tissue damage to the esophagus 104 caused by a cardiac ablation procedure being performed on the patient's heart 108. While the systems and methods disclosed herein primarily relate to the detection of esophageal tissue damage during a cardiac ablation procedure, in other embodiments and/or scenarios these systems and methods can be adapted to other uses, such as detecting tissue damage for diagnostic purposes before or after another procedure is performed.

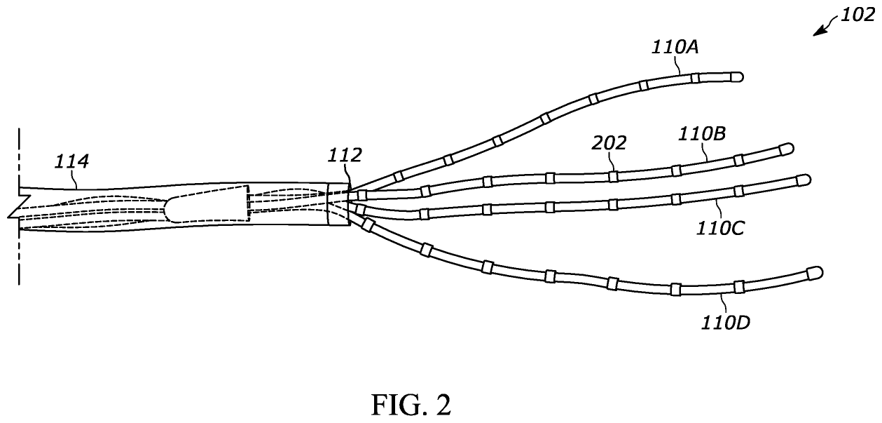

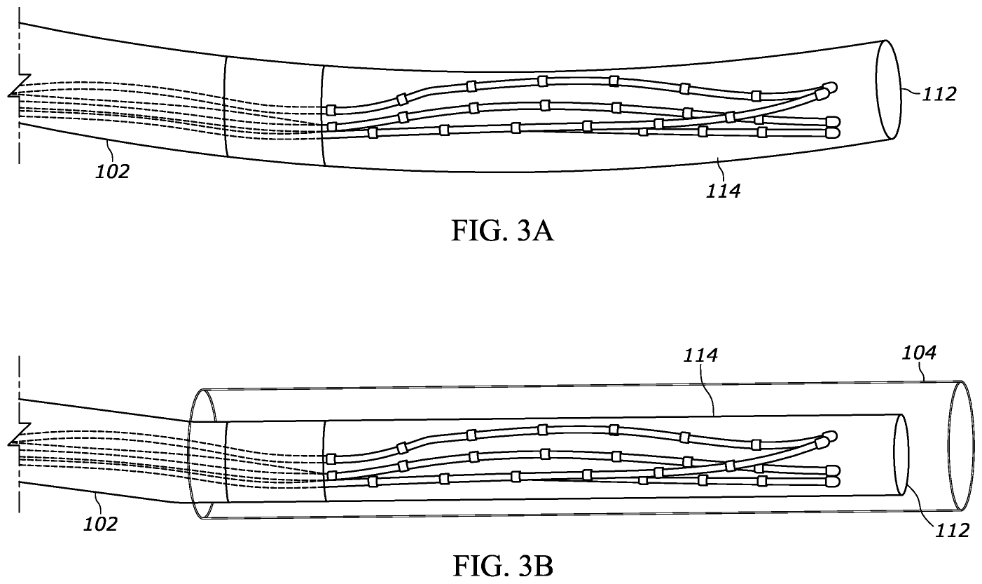

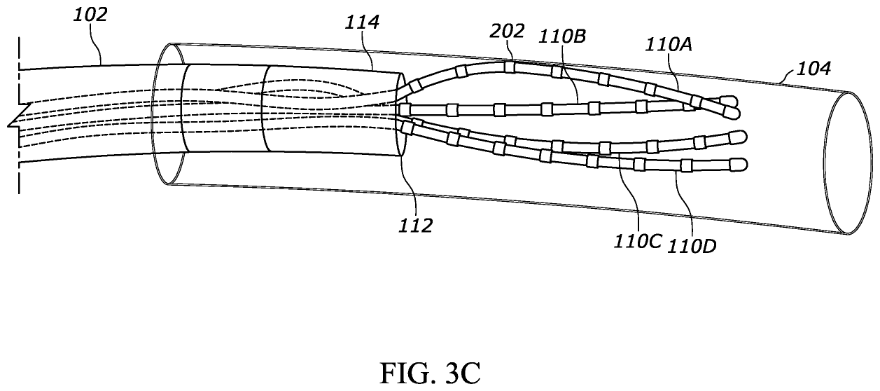

As shown in FIGS. 1 and 2, the probe 102 includes one or more tendrils 110A, 110B, 110C and 110D that can be selectively extended and retracted from an end 112 of a sheath 114 of the probe 102. FIG. 3A is an illustration of the probe 102 with the tendrils 110A-D retracted within the sheath 114. FIG. 3B is an illustration of the probe 102 positioned within an esophagus 104, which is depicted as a transparent tube in FIGS. 3B and 3C to better show the probe 102. When extended past the end 112 of sheath 114 (e.g., as shown in FIG. 3C) to monitor esophageal tissue for damage during a cardiac ablation procedure, the tendrils 110A-D expand outward (e.g., due to a spring wire 402 in each tendril 110A-C as shown in FIG. 4) such that a portion of each tendril 110A-D comes in contact along a different portion of the esophagus 104. Additionally and/or alternatively, tendrils 110A-C may be extended and retracted through holes in the side of the sheath 114. In some examples, the sheath 114 is omitted.

In some examples, the probe 102 and/or each of the tendrils 110A-D can be rotated with respect to the esophagus 104 to control the placement of the probe 102 and/or the tendrils 110A-D within and/or along the esophagus 104. For example, the tendrils 110A-D may be spaced apart around the esophagus 104, grouped near each other close to a cardiac ablation site, etc. In this way, a map of actual esophageal tissue damage (discussed below in more detail) for a portion of the esophagus 104 of interest (e.g., the side closest to the heart 108) can be characterized using the tendrils 110A-D.

As shown, each of the tendrils 110A-D (generally referred to in the singular as “tendril 110”) has a plurality of nodes (e.g., electrodes, transducers, etc.) 202 (see FIGS. 2 and 3C) spaced apart along a portion of the tendril 110. When a tendril 110 is deployed (i.e., extended), many of the tendril's nodes 202 will tend to come in contact with tissue along the esophagus 104. Spaced apart tendrils 110A-D as shown in FIGS. 1, 2 and 3C, together with spaced apart nodes 202 as shown in FIGS. 2 and 3C, result in a matrix or mesh of nodes 202 in contact with tissue at different locations/portions of the esophagus 104. The mesh of nodes 202 may be used (as discussed in more detail below) to characterize the tissue of all or a part of the esophagus 104 in proximity to a cardiac ablation site.

FIG. 4 is a cross-section of an example tendril 400 that may be used as each of one, some, or all of the tendrils 110A-D. The example tendril 400 illustrated in FIG. 4 includes a spring steel support 402 and four conductors 404A, 404B, 404C and 404D (e.g., wires, conductors, etc.) within a sheath 406. The conductors 404A-D are electrically coupled to respective nodes (one of which is designated at reference numeral 408) spaced apart along the tendril 400. Example nodes 408 for measuring bioimpedance are exposed metallic cylinders/rings/pads that at least partially encircle and contact the sheath 406, and are electrically coupled to the conductors 404A-D at one end. How the nodes 408 are coupled varies with measurement modality and multiple may be included. For temperature coupling, the sheath could be an electrical insulator, such as thermally conductive silicone; metallic rings or pads could be inserted into/around the sheath for bioimpedance nodes when electrical coupling is required; metal rings would also increase thermal conductivity when sensing temperature; for ultrasound, acoustic impedance needs to be minimized; for photoplethysmography (PPG), the sheath would need to be optically clear to the selected wavelengths; etc. The opposite end of the conductor 404A is coupled to a data analysis unit (e.g., the data analysis unit 120 shown in FIG. 1). As discussed in more detail below, the data analysis unit can transmit and/or receive signals via the node 408 by transmitting and/or receiving signals on the respective conductor 404A. Other nodes and conductors are similarly electrically coupled. While a specific number of tendrils 110A-D and nodes 202 are shown in FIG. 4, other embodiments have different numbers of tendrils 110A-D and/or nodes 202 arranged similarly and/or differently (e.g., with a different uniform node spacing, or with nodes non-uniformly spaced).

In the illustrated example of FIG. 4, the tendril 400 includes an example optical fiber 410. However, the optical fiber 410 may be implemented separately from the tendril 400 in other embodiments. The optical fiber 410 includes a plurality of fiber Bragg gratings (FBGs) (not shown) spaced apart along the optical fiber 410. The optical fiber 410 and the FBGs can be used to measure temperatures and/or strains at different locations along the esophagus 104. An FBG is a type of distributed Bragg reflector that reflects particular wavelengths of light and transmits the others. This is achieved by creating a periodic variation in the refractive index of the fiber core, which forms a wavelength-specific dielectric mirror. Hence, an FBG can be used as an inline filter to block certain wavelengths, or the FBG can be used as a wavelength-specific reflector. Because temperature changes the wavelength reflected by an FBG by changing physical properties of the FBG (e.g., the physical spacing between the periodic variations), the data analysis unit can determine temperature based on the wavelength of the light reflected by an FBG. It is not necessary that the optical fiber 410 and/or the FBGs contact tissue as long as the FBGs are close enough to sense the temperature(s) of the tissue(s) along the tendril 400. Moreover, the sheath 406 does not need to be transparent to light for the FBGs to sense the temperature(s).

Similarly, because strain on the optical fiber at an FBG changes the wavelength reflected by the FBG by changing physical properties of the FBG, the data analysis unit can measure strain based on the wavelength of the light reflected by the FBG. In some examples, the data analysis unit may use strain measurements at the FBGs along the optical fiber 410 to determine the position and/or orientation of the optical fiber 410. Additionally and/or alternatively, Rayleigh scattering with optical frequency domain reflectometry (OFDR) could be used, which provides high spatial resolution and does not require FBG inscription within in the optical fiber. The tendril can consist of three outer single core fibers or a single multi-core fiber used to measure the strain along its length. Example reconstruction methods that may be used to estimate the curvature of the fibers include Bishop frames, Frenet-Serret frames, parallel transport, a helical extension method, or constant curvature segmentation. For example, the position and orientation of the probe(s) can be determined using a variety of approaches alone or in combination, including but not limited to, X-ray fluoroscopy, intracardiac echocardiography, assigning split electrodes of the probe to form a 3D navigation system that utilizes impedance/dipole/RF current or magnetic sensors to display the esophageal probe in a 3D construct of the heart during catheter ablation.

In some examples, rigid supports are provided in connection with the FBGs to prevent strain from interfering with temperature measurements. For example, by encapsulating the fiber around the FBG site in a rigid metal/or other nonmetal tube. Further, because both strain, e.g., too much pressure on the esophagus, and thermal changes maybe equally concerning, a change in FBG signal may be used as an early alert regardless whether it is due to strain or temperature.

When the tendril 400 of FIG. 4 is deployed (i.e., extended from a sheath similar to the sheath 114), the spring steel support 402 forms the tendril 400 into an arc or curve that brings the nodes 408 into contact with tissue along a portion of the esophagus 104, and brings the FBGs into thermal proximity with the tissue along the portion of the esophagus 104.

An example tendril is approximately 40 mm long (20-100 mm range) with the smallest diameter possible (1-3 mm range). Nodes are as close together as possible (2-15 mm). An example 40 mm tendril has 8 nodes spaced 5 mm apart. Example sheaths have 3-12 tendrils. It's likely that the tendrils will not be evenly spaced since the heart is only on one side of the esophagus. The sheath/overall catheter diameter should be less than 10 mm (about 0.25″).

Returning to FIG. 1, the data analysis unit 120 is generally configured to introduce (e.g., transmit, convey, etc.) signals (e.g., electrical, acoustic, etc.) into esophageal tissue via the nodes 202, 408, and to receive (e.g., sense, measure, sample, etc.) signals (e.g., electrical, acoustic, etc.) present in or otherwise affected by esophageal tissue at the nodes 202, 408. The data analysis unit 120 processes these received signals to determine values of one or more characteristics. In particular, the data analysis unit 120 is configured to determine values of at least one characteristic that is indicative of actual (i.e., already existing) damage to esophageal tissue. By selecting a first node 202, 408 where a signal is introduced, and selecting a second node 202, 408 where a signal is received, the data analysis unit 120 can interrogate a property of the tissue of the esophagus between the two nodes. By systematically interrogating the properties of the tissue of the esophagus between various pairs of nodes, the properties of different, respective portions of the esophagus can be determined. As used herein, the term “unit” can, but does not necessarily, refer to only a single component or device. For example, the data analysis unit 120 may be a component of a multi-component device, an entire device, or a system that includes multiple devices. As more specific examples, the data analysis unit 120 may include separate signal generation and signal receiving/processing devices, or may include a single device that performs all of these operations.

In some scenarios, a physician may temporarily pause a cardiac ablation procedure while the data analysis unit 120 and probe 102 introduce and receive signals, in order to mitigate measurement interference when determining values of one or more esophageal characteristics. However, the data analysis unit 120 will primarily function during ablation to enable real-time assessment of esophageal tissue changes.

In some embodiments, the data analysis unit 120 includes a current source that introduces a current flow into esophageal tissue at a first node, and one or more receivers that measure a differential voltage across esophageal tissue between second and third nodes. In some examples, the current is introduced between the first node and a fourth node that surround the second and third nodes in a tetrapolar arrangement. In this embodiment, the data analysis unit 120 can determine a bioimpedance, resistivity, conductance, and/or other characteristic(s) of the esophageal tissue between the second and third nodes from the measured differential voltage. In some examples, the current flow is a constant, low-current signal for electrical safety, and to avoid any current-induced tissue heating. Because the bioimpedance, resistivity, conductance, etc. of esophageal tissue changes when esophageal tissue damage occurs, the bioimpedance, resistivity, conductance, etc. of the esophageal tissue determined by the data analysis unit 120 is indicative of actual esophageal tissue damage. Thus, when bioimpedance, resistivity, conductance, etc. changes (e.g., by a threshold amount), the data analysis unit 120 can detect the presence of actual esophageal tissue damage, and provide an indication (e.g., a visual and/or audio warning) of the damage to a user 130 (e.g., a surgeon performing a cardiac ablation procedure or an assistant) of a computing device 140 coupled to the data analysis unit 120.

Alternatively or additionally, in some embodiments, the data analysis unit 120 includes a sound wave generator that causes a first node (e.g., a transducer) to introduce a sound wave into esophageal tissue at the first node, and one or more receivers of the data analysis unit 120 measure sound waves that propagated through esophageal tissue to second and third nodes (e.g., other transducers). The data analysis unit 120 can determine shear-wave velocity (SWV) in the esophageal tissue between the second and third nodes, based on differences in arrival times of the sound waves at each node. Because the SWV of esophageal tissue changes when esophageal tissue damage occurs, the SWV of the esophageal tissue determined by the data analysis unit 120 is indicative of actual esophageal tissue damage. Thus, when SWV changes (e.g., by a threshold amount), the data analysis unit 120 can detect the presence of actual esophageal tissue damage, and provide an indication (e.g., a visual and/or audio warning) of the damage to the user 130 of the computing device 140. Alternatively or additionally, in some embodiments, the optical fiber 410 and its FBGs can be used by the data analysis unit 120 to determine temperatures at various points in and/or along the esophagus 104. Because esophageal tissue damage may occur in response to temperature increase, measured temperatures of the esophageal tissue can be used by the data analysis unit 120 to detect conditions likely to cause imminent esophageal tissue damage. Thus, when temperature changes (e.g., by a threshold amount), the data analysis unit 120 can detect the potential for esophageal tissue damage and provide an indication (e.g., a visual or audio warning) of the potential damage to the user 130 of the computing device 140. In some examples, actual damage indications (e.g., based on bioimpedance, conductivity, etc.) are provided separately from potential damage indications (e.g., based on temperature). However, the data analysis unit 120 may combine such indications into a single, multi-level indicator (e.g., green for no/low risk of damage, yellow for potential damage due to high temperature without any indication of actual damage, red for actual damage, etc.).

In some examples, the data analysis unit 120 uses a combination of bioimpedance, resistivity, conductance, and/or SWV, and possibly also temperature, to determine when esophageal tissue damage has occurred and/or may occur, and generates/provides an indication warning of actual and/or potential esophageal tissue damage to the user 130 of the computing device 140. For example, a change to a single value (e.g., bioimpedance) satisfying (e.g., exceeding) a threshold could trigger an indication. Additionally and/or alternatively, changes to multiple values of the same or different characteristics (e.g., bioimpedance and temperature) passing their respective thresholds may trigger an indication. Further, in some embodiments, the data analysis unit 120 may process values of one or more characteristics (at a single time, or for samples over a short window of time) to determine when to trigger an indication. For example, if the average of values of some characteristic over an area of esophageal tissue during a particular time window changes by a threshold amount, then the data analysis unit 120 may trigger an indication. The data analysis unit 120 (directly, or via one or more other devices/components) may provide indications to the user 130 of the computer device 140, and/or to another system communicatively coupled to the data analysis unit 120.

While an example monitoring system 100 is illustrated in FIG. 1, one or more of the structures and methods of FIG. 1 may be combined, divided, re-arranged, omitted, eliminated and/or implemented in other suitable ways. For example, the data analysis unit 120 and the computing device 140 may be combined, the computing device 140 may implement a portion of the data analysis unit 120 (e.g., a signal processing or analysis portion), etc. Further, the esophageal monitoring system 100 may include one or more structures and/or be configured to perform one or more methods in addition to, or instead of, those illustrated in FIG. 1 and/or described herein. Moreover, while the data analysis unit 120 is shown in FIG. 1 and described herein as a single unit, device or system, it may be separated into different portions. For example, signal generation, signal receiving, and signal processing may be done by different units, devices and/or systems.

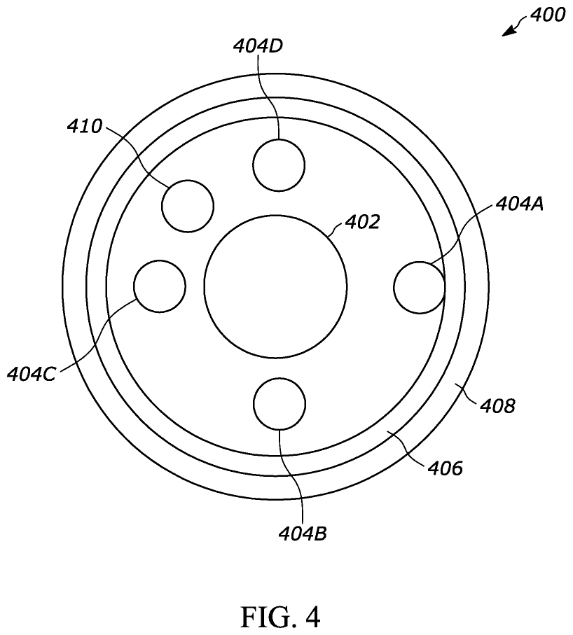

FIG. 5 is a block diagram of an example data analysis unit 500 that may be used as the example data analysis unit 120 of FIG. 1. To introduce signals into esophageal tissue via the nodes 202, 408, the example data analysis unit 500 includes any suitable number and/or type(s) of transmitters 502. Example transmitters 502 include one or more circuits capable of generating a direct current (DC) signal, an alternating current (AC) current signal, a pulse (which can be converted to an acoustic wave by a node 202, 408), etc.

To connect a transmitter 502 to a particular node of a particular tendril, the data analysis unit 500 includes any suitable number and/or type(s) of analog multiplexers 504. Control of the analog multiplexers 504 may be performed by a signal processor 506. Under the control of the signal processor 506, the analog multiplexers 504 can be configured such that a selected transmitter 502 can transmit a signal onto any particular conductor 508 of any particular tendril 510 to a respective node (not shown for clarity of illustration) of the tendril 510. Thus, the signal processor 506 can selectively determine the specific node(s) 202, 408 (and thus, the specific location(s) in an esophagus relative to the position of tendril 510) at which to introduce a desired signal into esophageal tissue.

To receive signals, the example data analysis unit 500 includes any number and/or type(s) of receivers 512. The example receivers 512 may include circuits capable of receiving, digitizing, sampling, capturing, etc. a signal (e.g., measuring a voltage) present in or otherwise affected by esophageal tissue. Under the control of the signal processor 506, the analog multiplexer 504 can be configured such that a selected receiver 512 can receive a signal on any particular conductor 514 of any particular tendril (e.g., the tendril 510 or another tendril 516) from a respective node (not shown for clarity of illustration) of the tendril 510 or 516. Thus, the signal processor 506 can selectively determine the specific node(s) 202, 408 (and thus, the specific location(s) in an esophagus relative to the position of tendril 516) from which to receive a signal present in or otherwise affected by esophageal tissue.

As shown in FIG. 5 and as described above in connection with FIG. 4, the conductors 508 and 514 may be electrically coupled to different nodes of the tendril 510. Alternatively, the conductors 508 and 514 may be electrically coupled to nodes of different tendrils 510 and 516.

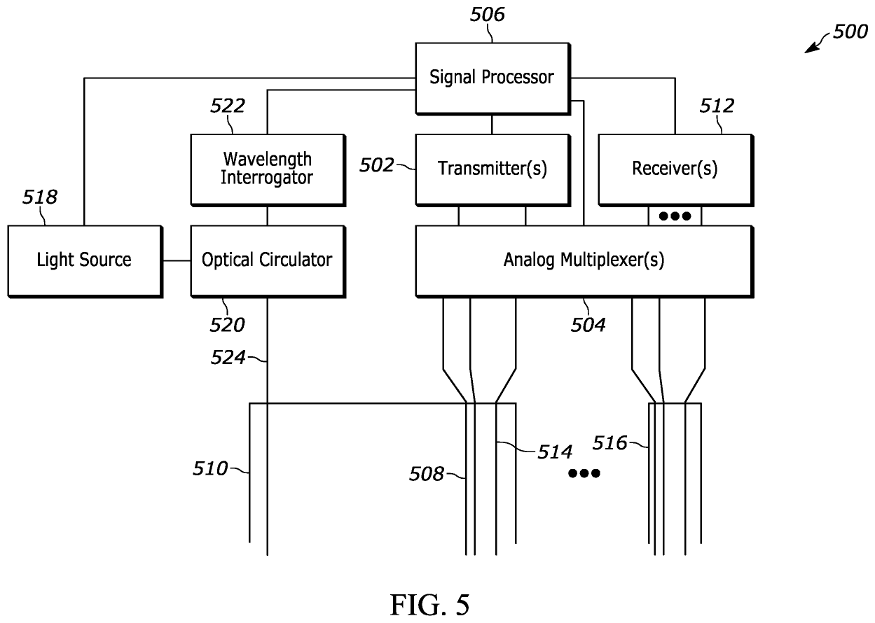

By controlling the transmitter(s) 502, the analog multiplexer(s) 504, and the receiver(s) 512, the signal processor 506 can interrogate particular portions of tissue of an esophagus by selecting nodes adjacent to the portions of esophageal tissue and measuring a signal change at or between the nodes. For example, the signal processor 506 can configure a transmitter 502 and the analog multiplexer 504 to introduce a current signal at a node 602 into esophageal tissue, as shown in FIG. 6, and then configure the analog multiplexer 504 to connect two receivers 512 to receive signals present in or otherwise affected by the esophageal tissue at nodes 604 and 606, respectively. In some examples, the current signal is introduced at the node 604 rather than the node 602. The signal processor 506 can process the signals received by the two receivers 512 to determine a differential voltage magnitude and/or phase at the esophageal tissue between the nodes 604 and 606. The signal processor 506 can then further process the voltage magnitude and/or phase to determine a value of a bioimpedance, resistivity, conductance, etc. of the esophageal tissue being interrogated. By systematically selecting the nodes 602, 604 and 606 and other combinations of nodes, the signal processor 506 can characterize a volume and/or portion of the esophagus.

Experimental data confirms that impedance measurements can be useful for identifying actual or potential tissue damage. For example, experiments that use sausage samples to simulate human tissue were run with 1100 Watts of heat power applied to each sausage samples. Applying probe signals having 50 mV peak-to-peak amplitude, 30 mV of DC offset, and 50 microamps of peak-to-peak current, at a frequency of 500 Hz, measurements were collected for the sausage samples over time. Collecting measurements once every 10 seconds, the normalized impedance measurements (mean and standard deviation) were as follows:

TABLE 1

| | Sample 0 | Sample 1 | Sample 2 | Sample 3 |
|---|---|---|---|---|
| | (mean; | (mean; | (mean; | (mean; |
| Measurement | standard | standard | standard | standard |
| number | dev) | dev) | dev) | dev) |
| 1 | 1.1; 0.1 | 1.3; 0.1 | 1.3; 0.1 | 1.4; 0.1 |
| 2 | 1.7; 0.4 | 13.9; 1.2 | 1.7; 0.2 | 2.1; 0.3 |
| 3 | 2.9; 1.5 | 79.9; 130.0 | 4.9; 2.4 | 3.9; 2.3 |
| 4 | 971.0; 724.4 | — | — | — |

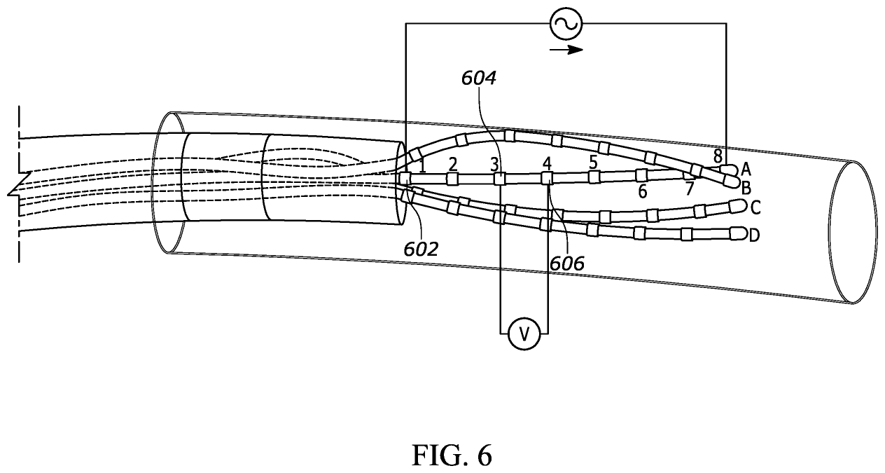

The values of the characteristic determined by the signal processor 506 can be used, for example, along with the known (relative) locations of the nodes that the analog multiplexer(s) 504 selected to receive the respective signals, to provide a map/visualization of the electrical properties of an area or volume of esophageal tissue. FIG. 7 depicts an example interpolated esophageal tissue conductivity visualization 700 that highlights an esophageal tissue region (shown darker in FIG. 7, e.g., region 702) with low conductivity (i.e., actual esophageal tissue damage), which may be due to excessive heating from a cardiac ablation procedure. Lighter areas (e.g., in a region 704) have higher or normal conductivities and, thus, have not yet experienced actual tissue damage. Alternatively, rather than depicting areas with shades of black and white, as shown in FIG. 7, colors may be used. For example, areas with low conductivity (e.g., in the region 702) may be depicted with red, and areas with normal conductivity (e.g., in the region 704) may be depicted with blue, with a continuum of colors (e.g., oranges, yellows, etc.) used in between. The visualization 700 is a flattened representation of a round, tubular esophagus, and each intersection within the visualization 700 (e.g., A1, B1, A2, etc.) may correspond to a different node.

In some examples, the signal processor 506 presents a first visualization 700 of pre-procedure values of the characteristic, and a second visualization 700 of during-procedure values of the characteristic to aid in the identification of actual esophageal tissue damage due to the procedure. In other examples, the visualization 700 itself presents the difference of the values between pairs of nodes (i.e., the change from the pre-procedure values). In some examples, when one of these differences in value satisfy (e.g., exceed) a pre-determined threshold, the signal processor 506 presents an indication (e.g., a visual and/or audio warning) of actual esophageal tissue damage at the computing device 140 and/or another device coupled to the data analysis unit 500. Alternatively, the signal processor 506 may present such an indication based on one or more other criteria (e.g., the average change in value for two adjacent areas, such as between nodes B2 and C2, exceeding a threshold, etc.). The pre-determined threshold may be determined experimentally. In some examples, any change from baseline would indicate a change in tissue state and the physician could/should be alerted that a change is actively occurring. The baseline may be determined using a long-duration (e.g., 10 s) average or longer filter from which a baseline threshold could be determined and then used to detect short-time (<10 s) changes that exceed the threshold. Although detection can be based on absolute values, the detection may be based on spatiotemporal gradients which are relative to adjacent sensors/pixels in space or to all sensors/pixels in space and time. Relative changes >10%, 20%, etc. can be set as alert thresholds by the operator. Similar visualizations can be generated for different frequencies (such as 1, 10, 100 or 1,000 kHz), and/or show other variables, such as phase shift, peak impedance and/or compare measurements performed at different frequencies. For example, a signal processing algorithm may extract these features from the signal. They could then be tracked over time and/or graphed as in FIG. 7. The previously mentioned deviation from baseline detector could also be used to detect changes in these measurements in order to alert the physician to changing tissue properties using a graphical, audio or tactile alert.

In some examples, tendrils are not equally spaced about the esophagus. In such examples, the vertical lines representing the tendrils A-F in FIG. 7 can be moved right and left in the visualization 700 to better reflect their actual locations in the esophagus, as shown in visualization 800 of FIG. 8. In some examples, the catheter is designed to allow some course control of tendril placement and expected deployment, but once deployed their true position may need to be directly measured using electromagnetic 3D tracking (e.g., x-ray or magnetic triangulation), estimated from the tendril strain (i.e., measured using FBGs) or an assumption can be made based on experience/experimental data.

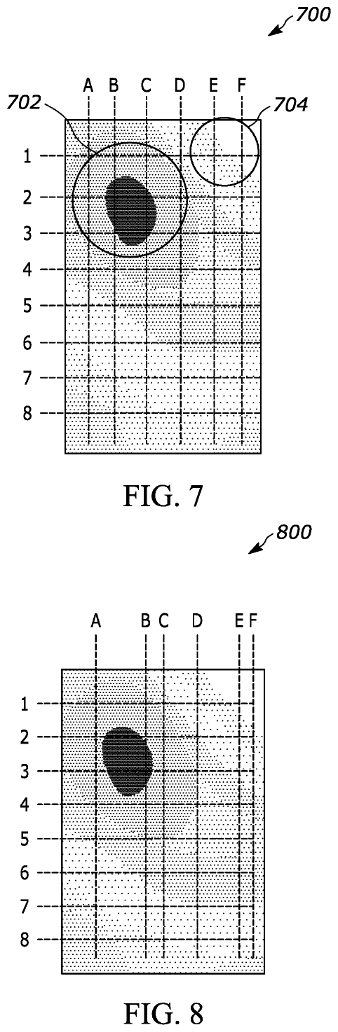

In another example, shown in FIG. 9, the signal processor 506 configures a transmitter 502 and the analog multiplexer 504 to introduce a sound wave signal at a transducer node 902 into esophageal tissue, and then configures the analog multiplexer 504 to connect two receivers 512 to receive time-domain, sound wave signals in the esophageal tissue via transducer nodes 904 and 906, respectively. In some examples, the sound wave signal is introduced at the node 904 rather than the node 902, and is received only via node 906. The signal processor 506 can process the signals received by the two receivers 512 to determine a difference (At) between the times at which the sound wave signal is received at the nodes 904 and 906. If the signal processor 506 causes the sound wave signal to be introduced at node 904, then At is the difference between the arrival time at node 906 and the known time at which the sound wave signal was introduced via node 904. Knowing the distance (As) between the nodes 904 and 906, the signal processor 506 can determine the SWV of the sound wave in the esophageal tissue between the nodes 904 and 906. If the sound wave signal is introduced at node 902, for example, the signal processor 506 may determine the SWV between the nodes 904, 906 by correlating the signals received via the nodes 904, 906, because the same shear-wave signature should be present in both signals, but shifted in time.

To determine the time difference Δt, each received signal may be shifted, multiplied and summed with themselves and/or the introduced sound wave to form auto- and cross-correlation signals. This may be expressed mathematically, as shown in EQN (1) below, where y is the correlation, x represents the introduced signal, h is the received signal, M is the number of samples in the received signal, and i is the position (shift) of the received signal h relative to the introduced signal that is currently being calculated.

$$y [ i ] = {\sum }_{j = 0}^{M - 1} h [ j ] x [ i - j ] \qquad EQN ( 1 )$$

Movement of the sound wave in esophageal tissue is affected by physical structures of the esophageal tissue (e.g., cells). Mechanical deformation (e.g., compression) of the cells occurs due to the pressure of the sound wave travelling through the tissue. Stiffer cells (tissue) result in faster propagation of the shear wave and vice versa. The amount of signal shift required to find the maximum correlation therefore corresponds to the mean velocity of the shear wave, due to esophageal tissue stiffness. Cells that are less elastic (e.g., damaged) result in less shift and, thus, a higher SWV. If the transmission time of each sound wave is accurately controlled, the signal processor 506 can determine the shift and, thus, can determine the velocity of transverse waves propagating in the esophageal tissue (i.e., SWV).

In some examples, the sound wave is implemented using ultrasound, where each node introduces a pulsed acoustic wave (e.g., 8 sinusoidal cycles at 5 MHz repeated at a 10 KHz interval). As time passes, the introduced acoustic wave propagates perpendicular to the tissue surface deeper into the tissue. Variations in tissue properties results in variation in acoustic impedance, which in turn results in reflections of a small amount of the introduced wave's energy. This reflected acoustic energy can be detected by the node as a time-domain voltage signal, where the time represents depth within the tissue.

If sufficient acoustic energy is introduced, the resulting compression wave can initiate a shear wave that travels laterally (parallel to the surface of the skin). As the shear wave propagates it will displace the tissue perpendicular to the surface of the skin slightly.

This propagating displacement can be detected using ultrasound as the tissue motion will also change the reflected acoustic energy, which manifests as a disturbance in the time-domain voltage signal. By measuring the elapsed time between the introduced ultrasound energy and the associated disturbance in the received voltage signal, the SWV can be determined.

By systematically selecting the nodes 902, 904 and 906 (or nodes 904 and 906, if introducing the sound wave signal via node 904), the signal processor 506 can characterize an area or volume of the esophagus. The SWVs determined by the signal processor 506 can be combined to provide a visualization of esophageal tissue elasticity (e.g., similar to the conductivity visualization 700 of FIG. 7). A region that exhibits an increasing SWV is indicative of increasing stiffness (i.e., lowering elasticity) and, thus, may represent an area where esophageal tissue damage is occurring due to excessive heating from a cardiac ablation procedure. As discussed above with reference to FIG. 7, the signal processor 506 may generate one or multiple visualizations (e.g., showing absolute values and/or changes in values), and may apply any of various criteria (e.g., thresholds for individual absolute or difference values, or thresholds for metrics calculated based on multiple absolute or difference values measurements, etc.) depending on the embodiment.

In some examples, instead of a static array of single-element transducers mounted on the tendrils as shown in FIG. 9, a two-dimensional (2D) or three-dimensional (3D) phased-array of sufficiently spaced apart transducers (e.g., piezo-electric transducers (PZT) or capacitive, micro-machined, ultrasonic transducers (CMUT)) could be used to either electronically or mechanically scan an esophagus at the same time to generate 2D or 3D maps of esophageal elasticity in order to detect thermally-inducted esophageal tissue damage.

Returning to FIG. 5, to measure the blood perfusion of the esophageal tissue of an esophagus, the data analysis unit 500 includes a light source 518 and an optical circulator 520, and is able to measure the intensity of a received optical signal. To measure tissue perfusion along an optical fiber 524, the light source 518 transmits light into the optical fiber 524 via the optical circulator 520. The light travels down the optical fiber 524. The end of each fiber is either aligned to terminate in contact with the tissue or a small mirror included (or fiber cut so that the mechanism of total internal reflection can be used to redirect the light) so that the incident light is coupled into the tissue adjacent to the tendril. A series of mirrors (e.g., narrow-band, single-frequency or dielectric mirrors) that only reflect certain wavelengths of light could be placed in series along the tendril (forming specific nodes) the received light after exiting the circulator 520 would be subjected to another series of similar mirrors or selective filters forming an optical demultiplexer and allow the intensity of each narrow band of wavelengths originating from each node to be measured. As the light penetrates the tissue, scattering or absorption occurs, and some portion of the reflected component will reenter the optical fiber and return to the optical circulator after which its intensity can be measured using a series of detectors. Instead of individual detectors, an array or matrix of detectors may also be used, such as a photoplethysmographic image (PPGi) sensor/camera. In other implementations, a PPGi sensor could be directly integrated at the measurement site with the tendril or catheter, so that no fiber optic wave guide is necessary. Regardless of the actual implementation, since tissue perfusion changes during each heartbeat, the intensity of the received wavelengths will also be modulated over each heartbeat. If tissue damage occurs due to ablation its perfusion will also be reduced.

To measure temperatures of esophageal tissue of an esophagus, the data analysis unit 500 includes the light source 518, the optical circulator 520, and a wave length interrogator 522. To measure temperatures along an optical fiber 524, the light source 518 transmits broad spectrum light into the optical fiber 524 via the optical circulator 520. The light travels down the optical fiber 524. When the light reaches each FBG, one wavelength of light is reflected back toward the data analysis unit 500, and the remaining light travels on to the next FBG. It is understood that, as used herein, a “wavelength” may refer to a relatively narrow range of wavelengths, rather than precisely one wavelength. The wavelength that is reflected depends on the physical layout of the FBG. When the FBG is exposed to heating (e.g., due to cardiac ablation) the FBG physically expands and, thus, the wavelength of light that is reflected changes. The wavelength of the reflected light can be determined by the wavelength interrogator 522. A change in wavelength detected by the wavelength interrogator 522 can be used by the signal processor 506 to determine the temperature of the FBG that is in intimate or close proximity to the esophageal tissue. The temperature at the other FBGs of the fiber optic 524 can be likewise determined. The signal processor 506 can combine the temperatures to map a volume of esophageal tissue (e.g., similar to the visualization 700 of FIG. 7) and provide a visualization of the esophageal tissue's temperatures. A region that exhibits increasing temperature is indicative of potential esophageal tissue damage. In some examples, the signal processor 506 presents a first visualization of pre-procedure temperatures (or temperatures recorded several seconds or minutes ago), and a second visualization is presented of during-procedure (current) temperatures to aid in the identification of potential esophageal tissue damage. In some examples, the signal processor 506 presents a difference of two such visualizations, where areas of difference correspond to potential esophageal tissue damage. In some examples, when such differences satisfy (e.g., exceed) a pre-determined threshold, the signal processor 506 presents an indication (e.g., a visual and/or audio warning) of potential esophageal tissue damage at the computing device 140 and/or another device coupled to the data analysis unit 500. The pre-determined threshold may be determined experimentally.

While an example manner of implementing the data analysis unit 120 of FIG. 1 is illustrated in FIG. 5, one or more of the structures and methods illustrated in (or discussed in connection with) FIG. 5 may be combined, divided, re-arranged, omitted, eliminated and/or implemented in any other suitable way. For example, the transmitters 502, the analog multiplexers 504, the receivers 512, and the signal processor 506 may be implemented by separate devices, units, systems, etc. For instance, the transmitters 502, the analog multiplexers 504, and the receivers 512, may be implemented by one or more off-the-shelf systems, and the signal processor 506 may implemented by the computing system 150 while communicatively coupled to the off-the-shelf system(s). Further, the transmitter(s) 502, the analog multiplexer 504, the signal processor 506, the receiver(s) 512, the light source 518, the optical circulator 520, the wavelength interrogator 522 and/or, more generally, the data analysis unit 500 may be implemented by hardware, software, firmware and/or any combination of hardware, software and/or firmware. Thus, for example, any of the transmitter(s) 502, the analog multiplexer 504, the signal processor 506, the receiver(s) 512, the light source 518, the optical circulator 520, the wavelength interrogator 522 and/or, more generally, the data analysis unit 500 could be implemented by one or more of a digital or analog circuit, logic circuit, programmable processor, programmable controller, graphics processing unit (GPU), digital signal processor (DSP), application specific integrated circuit (ASIC), programmable logic device (PLD), field programmable gate array (FPGA), and/or field programmable logic device (FPLD). In some examples, the signal processor 506 is implemented by the computing device 140 of FIG. 1. In some purely software implementations, the signal processor 506 and/or the data analysis unit 500 execute instructions stored on a non-transitory, machine-readable storage medium, device or disk (e.g., a hard disk drive (HDD), a solid-state drive (SDD), a flash memory, a digital versatile disk (DVD), a compact disc (CD), a Blu-ray disk, a cache, a flash memory, a read-only memory (ROM), a random access memory (RAM), etc.)

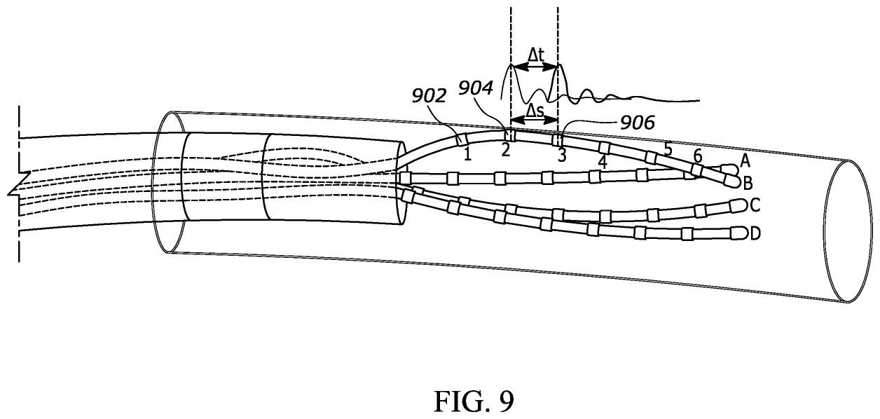

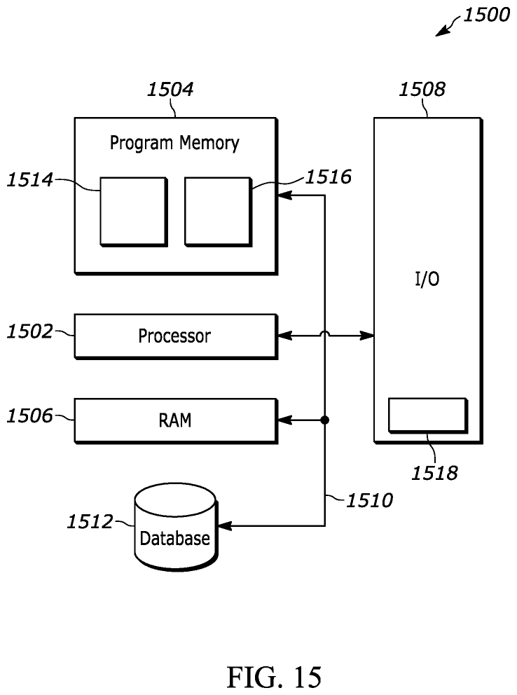

FIG. 10 is a flowchart 1000 representative of an example process that may be implemented by the data analysis unit 120 or 500. The process may be performed by a processor (e.g., the processor 1502 of FIG. 15) executing a program. The program may be embodied in software and/or instructions stored on a tangible, non-transitory, machine-readable storage medium, device or disk (e.g., a CD, an HDD, a DVD, a Blu-ray disk, a cache, a flash memory, a ROM, a RAM, etc.), for example. Further, although an example program is described with reference to the flowchart 1000 illustrated in FIG. 10, many other methods may instead (or also) be implemented by the data analysis units 130, 500. For example, the order of execution of the blocks may be changed, and/or some of the blocks described may be changed, eliminated, or combined. Additionally, or alternatively, any or all of the blocks may be implemented by one or more hardware circuits (e.g., discrete and/or integrated analog and/or digital circuitry, an ASIC, a PLD, an FPGA, an FPLD, a logic circuit, etc.) structured to perform the corresponding operation without executing software or instructions.

The program of FIG. 10 begins with the signal processor 506 selecting a source node at which a signal will introduced into esophageal tissue, and one or more measurement nodes at which signals will be received (block 1002). In some examples, one of the measurement nodes is also the source node. The signal processor 506 causes the analog multiplexer 504 to connect a transmitter 502 to the source node, and to connect the measurement node(s) to receivers 512 (block 1004). The signal processor 506 causes the transmitter(s) 502 to introduce a signal into esophageal tissue at the source node (block 1006) and the receiver(s) 512 to receive signals present in or otherwise affected by the esophageal tissue at the measurement node(s) (block 1008).

The signal processor 506 processes the signals received via the measurement node(s) and receiver(s) 512 to determine a value of a characteristic indicative of actual esophageal tissue damage for the esophageal tissue between the measurement node(s) (block 1010). The signal processor 506 updates an esophageal tissue characterization map, such as the example map of FIG. 7, based on the determined value of the characteristic (block 1012), and presents the updated esophageal tissue characterization map (block 1014), e.g., to a physician.

The signal processor 506 controls the light source 518 and the wave length interrogator 522 to measure temperatures of esophageal tissue along the esophagus (block 1016). Based upon the measured temperatures, the signal processor 506 presents an updated temperature map (block 1018). In some examples, blocks 1016 and 1018 are performed in parallel with blocks 1002 to 1014.

If any values of one or more esophageal tissue characteristics have changed by more than a respective threshold amount (block 1020), the signal processor 506 presents an indication (e.g., a visual and/or audio warning) of actual esophageal tissue damage (block 1022).

If any temperatures have changed by more than a threshold amount (block 1024), the signal processor 506 presents an indication (e.g., a visual and/or audio warning) of potential esophageal tissue damage (block 1026). Control then returns to block 1002 to continue introducing into and receiving signals from esophageal tissue.

In some examples, block 1026 is omitted, and/or the warning of actual esophageal tissue damage is presented at block 1020 only when both (1) one or more values of an esophageal tissue characteristic have changed by more than a first threshold amount, and (2) one or more temperatures have changed by more than a second threshold amount.

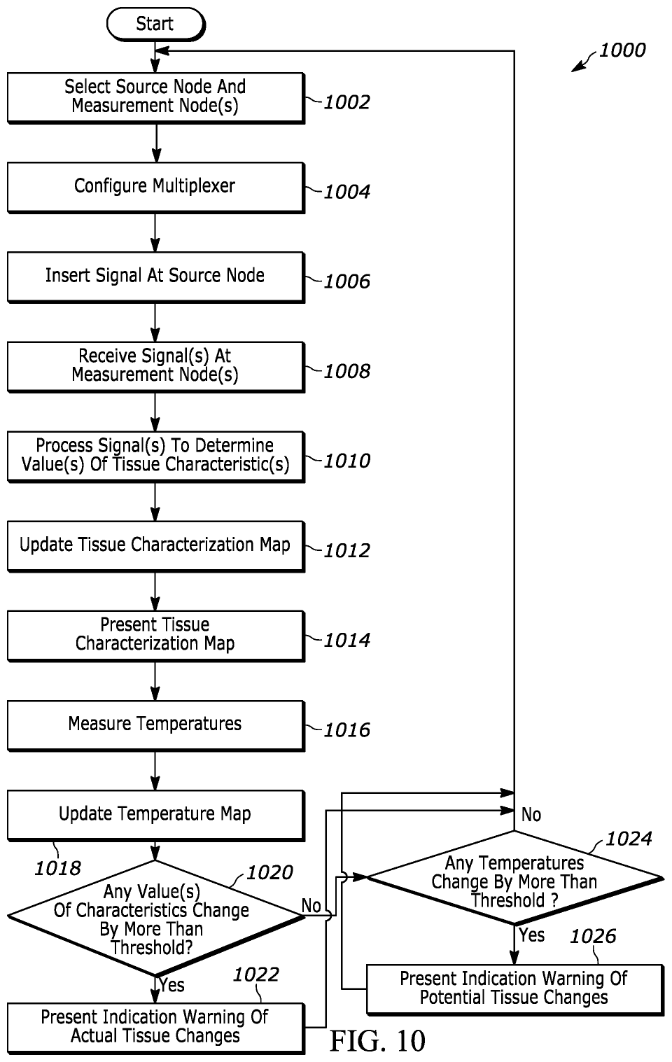

FIG. 11 is a flowchart 1100 representative of another example process that may be implemented by the data analysis unit 120 or 500. The process may be performed by a processor (e.g., the processor 1502 of FIG. 15) executing a program. The program may be embodied in software and/or instructions stored on a tangible, non-transitory, machine-readable storage medium, device or disk (e.g., a CD, an HDD, a DVD, a Blu-ray disk, a cache, a flash memory, a ROM, a RAM, etc.), for example. Further, although an example program is described with reference to the flowchart 1000 illustrated in FIG. 10, many other methods may instead (or also) be implemented by the data analysis units 130, 500. For example, the order of execution of the blocks may be changed, and/or some of the blocks described may be changed, eliminated, or combined. Additionally, or alternatively, any or all of the blocks may be implemented by one or more hardware circuits (e.g., discrete and/or integrated analog and/or digital circuitry, an ASIC, a PLD, an FPGA, an FPLD, a logic circuit, etc.) structured to perform the corresponding operation without executing software or instructions.

The method of FIG. 11 begins with deploying a probe in a patient's esophagus, wherein the probe includes a sheath and a plurality of tendrils extendible by a user from an end of the sheath (block 1102). The plurality of tendrils are configured to contact respective esophageal tissue at different locations in the esophagus when extended from the end of the sheath, and each tendril of the plurality of tendrils includes a plurality of nodes spaced apart along the tendril. Each node of the plurality of nodes is configured to introduce signals into esophageal tissue and/or receive signals affected by esophageal tissue.

The signal processor 506 introduces a first signal into esophageal tissue using a first node (block 1104). The signal processor 506 then determines, during a cardiac ablation procedure and based on a second signal received using a second node, a value of a characteristic (e.g., conductance, resistance, reactance, bioimpedance, etc.) indicative of actual damage to esophageal tissue of the patient's esophagus in proximity to a cardiac ablation site and between the second node and a third node (block 1106).

The signal processor 506 monitors for actual esophageal tissue damage during the cardiac ablation procedure by determining, based (at least in part) on the value that was determined at block 1106, whether actual esophageal tissue damage has occurred (block 1108). At some point during the cardiac ablation procedure, when determining that actual esophageal tissue damage has occurred, the signal processing 506 provides an indication of actual esophageal tissue damage (block 1110).

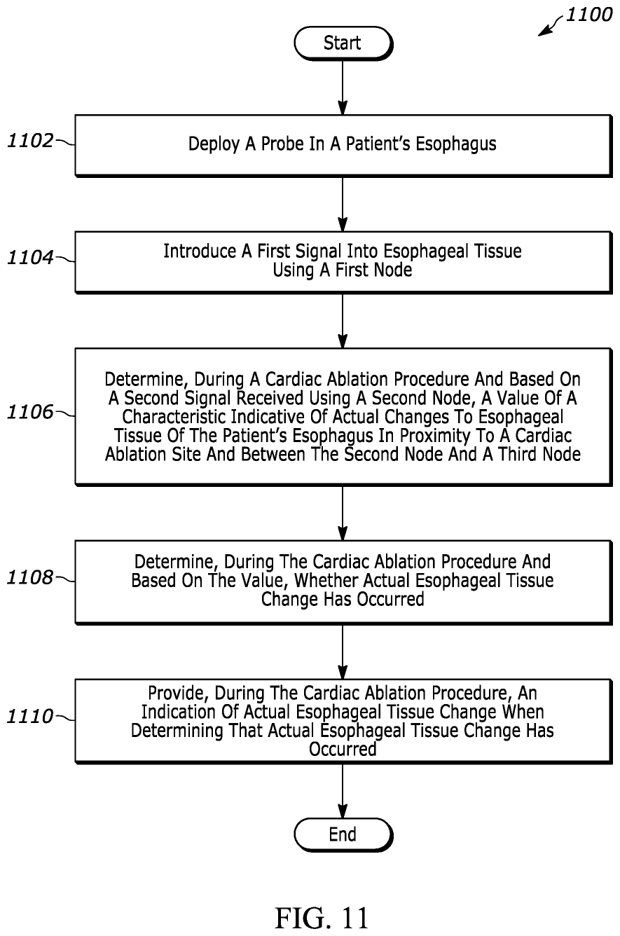

In some examples, and for any of the embodiments discussed above in connection with FIGS. 1 through 11, at least one tendril includes a mechanism that enables the tendril to be rotated within an esophagus and then arched in a way that can be used to retract the esophagus away from a cardiac ablation site. For example, FIG. 12A is a diagram illustrating an example soft robotics actuator 1202 that may be used to arch a tendril 1104. The soft robotics actuator 1202 includes an air cavity 1206 into which pressurized air may be introduced. The air cavity 1206 is more flexible than the rest 1208 of the tendril 1104 and, thus, when air in the air cavity 1206 is pressurized, the air cavity will expand and force the rest 1208 of the tendril 1104 to arch, as shown in FIG. 12B. The air cavity 1206 can be unpressurized to straighten the tendril 1204 to its original state, as shown in FIG. 12A, for removal from an esophagus.

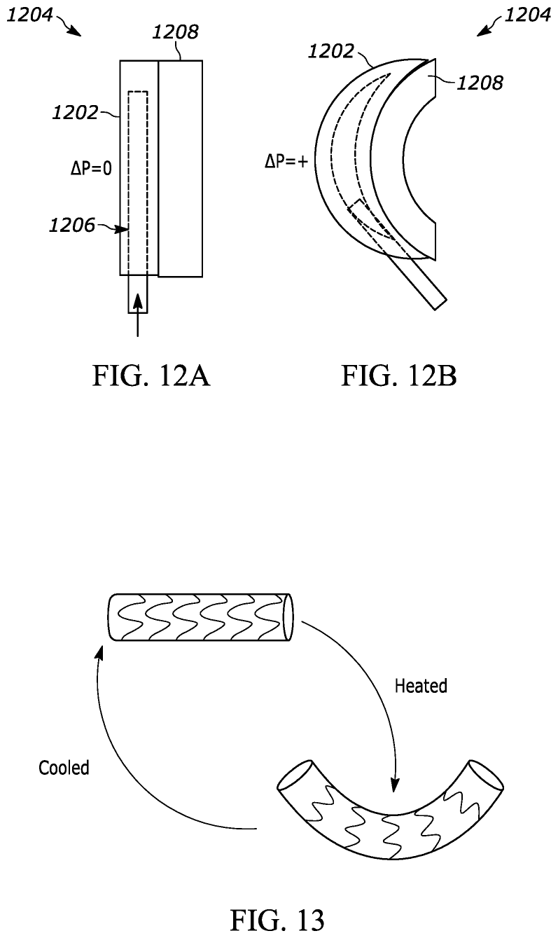

In another example, shown in FIG. 13, a shape memory alloy is used that, when heated or cooled, changes its shape. At a lower temperature (e.g., room or body temperature), the alloy can be deformed into any shape (e.g., straightened). When the alloy is heated, the alloy goes through a transformation where the alloy “remembers” its original shape (e.g., bent). The alloy may be heated using an electrical current. Additionally and/or alternatively, a bimetallic strip, which when heated will deform in one direction and return when cooled, may be used.

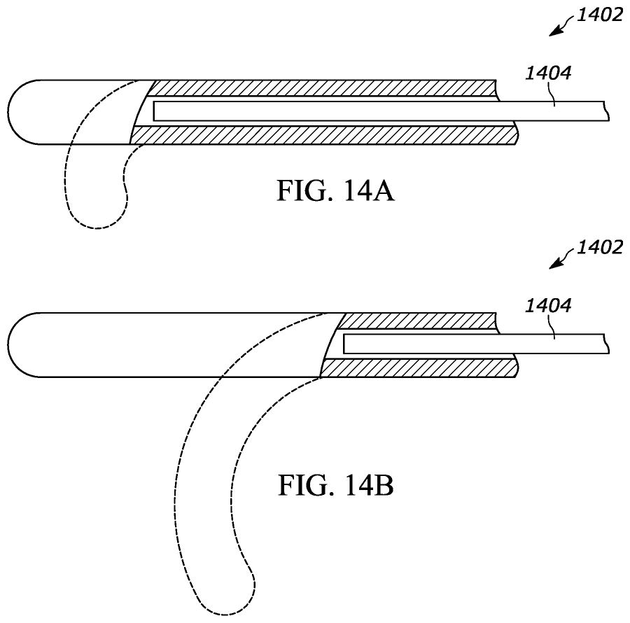

In yet another example, shown in FIG. 14A, a pre-strained tendril 1402 has an inner core 1404 that can be removed/withdrawn to allow the pre-strained tendril 1402 to bend, as shown in FIG. 14B. The inner core 1404 can be reinserted to straighten the tendril 1402 to its original state, as shown in FIG. 14A, for removal from an esophagus.

In yet another example, an actuator wire running adjacent to, but only attached at the tip of an inner core could be subjected to a longitudinal force, which would induce a bending moment in the inner core.

For all these methods of inducing a curvature in the catheter in order to move the esophagus away from the wall of the left atrium, a method may be need to be employed to anchor one or both ends of the catheter to enable or enhance the esophageal-left atrial wall spacing. One method to achieve this utilizes an inflatable balloon on one or both ends of the catheter incorporating the curvature mechanism that serve as anchor points. Should two balloons be utilized, a vacuum could be applied to the lumen of the esophagus between the balloons, which would draw the esophageal wall into direct contact with the catheter and away from the heart. In addition to moving the esophagus away from the ablation site, the vacuum would also improve the electrical, thermal and/or optical contact between the nodes and the esophageal wall, to improve the measured signals. In addition to the vacuum, the curvature mechanism could then be activated to further increase clearance between the esophagus and heart.

Improving the thermal, electrical, and/or acoustic coupling between the catheter tendrils and esophagus wall improves the robustness of the measurements of tissue properties. This can be achieved by introducing a coupling medium, for example, a saline solution. Additionally, a vacuum could be subsequently applied to draw the tissue into intimate contact with the catheter tendrils, trapping a small amount of fluid between the measurement node and esophageal tissue. The systems described herein could be further enhanced to introduce this medium through the catheter and then remove it using the same conduit or a second conduit using a vacuum.

Because tissue damage can be caused through excessive heating (i.e., during cardiac ablation using electric current) or cooling (i.e., during cryoablation) of the tissue during the ablation procedure, the liquid or gas medium could be heated or cooled before continuous or intermittent ejection onto the esophageal tissue in order to help control its temperature. Excess fluid or gas could be removed by the same or second conduit either under its own pressure or using a vacuum. In certain implementations, a control system could be used to introduce the heated or cooled medium while maintaining the esophageal lumen pressure below atmospheric pressure either continuously or intermittently, so that the esophageal wall remains in intimate contact with the catheter, but the heated or cooled medium can still be drawn across the surface of the esophageal wall. This may take the form of a catheter that injects the medium at the base of the tendril under low pressure and then extracts the medium at the tip of the tendril using a low vacuum, or vice versa.

FIG. 15 is a block diagram of an example computing system 1500 that may be used to, for example, implement all or part of the signal processor 506, the data analysis units 130, 500, and/or the computing device 140. The computing system 1500 may be, for example, a server, a personal computer, a laptop, a tablet, a workstation, or any other type of computing device or system.

The computing system 1500 includes a processor 1502, a program memory 1504, a RAM 1506, and an input/output (I/O) circuit 1508, all of which are interconnected via an address/data bus 1510. It should be appreciated that although FIG. 15 depicts only one processor 1502, the computing system 1500 may include multiple processors 1502. The processor 1502 of the illustrated example is hardware, and may be a semiconductor based (e.g., silicon based) device. Example processors 1502 include a programmable processor, a programmable controller, a GPU, a DSP, an ASIC, a PLD, an FPGA, an FPLD, etc.

The program memory 1504 may include any number and/or type(s) of non-transitory, volatile and/or non-volatile, machine-readable storage medium, devices or disks storing software or machine-instructions that may be executed by the processor 1502 to implement all or part of the signal processor 506, the data analysis units 130, 500, and/or the computing device 140. However, different portions of the signal processor 506, the data analysis units 130, 500, and/or the computing device 140 may be implemented by other computing systems. The software or machine-readable instructions may be stored on separate non-transitory, machine-readable storage mediums, devices or disks, and/or at different physical locations.

Example memories 1504, 1514, 1516 include any number or type(s) of volatile or non-volatile, non-transitory, machine-readable storage medium, devices or disks, such as a semiconductor memories, magnetically readable memories, optically readable memories, an HDD, an SSD, a ROM (e.g., a ROM 1514), a RAM (e.g., a RAM 1516), a redundant array of independent disks (RAID) system, a cache, a flash memory, or any other storage medium, device or disk in which information may be stored for any duration (e.g., permanently, for an extended time period, for a brief instance, for temporarily buffering, for caching of the information, etc.).

As used herein, the term non-transitory, machine-readable medium is expressly defined to include any type of machine-readable storage device and/or storage disk and to exclude propagating signals and to exclude transmission media.

In some embodiments, the processor 1502 may also include, or otherwise be communicatively connected to, a database 1512 or other data storage mechanism (one or more hard disk drives, optical storage drives, solid state storage devices, CDs, CD-ROMs, DVDs, Blu-ray disks, RAID, etc.).

The processing platform 1500 of FIG. 15 includes one or more communication interfaces such as, for example, a network interface 1518 and/or an input/output (I/O) interface 1508. The communication interface(s) enable the processing platform 1500 of FIG. 15 to communicate with, for example, another device (e.g., the data analysis units 130, 500), system (e.g., the computing device 140), host system, a datastore or database, or any other machine.

Although FIG. 15 depicts the I/O circuit 1508 as a single block, the I/O circuit 1508 may include a number of different types of I/O circuits or components that enable the processor 1502 to communicate with peripheral I/O devices and/or other computing systems. Example interface circuits 1508 include a universal serial bus (USB) interface, a Bluetooth® interface, a near field communication (NFC) interface, an infrared interface and/or a PCI express interface. The peripheral I/O devices may be any desired type of I/O device such as a keyboard, a display (a liquid crystal display (LCD), a cathode ray tube (CRT) display, a light emitting diode (LED) display, an organic light emitting diode (OLED) display, an in-place switching (IPS) display, a touch screen, etc.), a navigation device (a mouse, a trackball, a capacitive touch pad, a joystick, etc.), a speaker, a microphone, a printer, a button, a communication interface, an antenna, the data analysis unit 120, the probe 102, the data analysis unit 500, the transmitter(s) 502, the analog multiplexer 504, the receiver(s) 512, the light source 518, the wavelength interrogator 522, etc.

The I/O circuit 1508 may include a number of different network interfaces 1518 that enable the computing system 1500 to communicate with other computing systems, such as the computing system 1500, that implement other portions of the signal processor 506, the data analysis units 130, 500, and/or the computing device 140 via, e.g., a network such as the Internet. The network interface 1518 may be a wireless fidelity (Wi-Fi) transceiver, a cellular transceiver, an Ethernet network transceiver, an asynchronous transfer mode (ATM) network transceiver, a digital subscriber line (DSL) modem, a dialup modem, a satellite transceiver, a cable modem, etc.

Use of “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise. A device or structure that is “configured” in a certain way is configured in at least that way, but may also be configured in ways that are not listed.

Further, as used herein, the expressions “in communication,” “coupled” and “connected,” including variations thereof, encompasses direct communication and/or indirect communication through one or more intermediary components, and does not require direct mechanical or physical (e.g., wired) communication and/or constant communication, but rather additionally includes selective communication at periodic intervals, scheduled intervals, aperiodic intervals, and/or one-time events. The embodiments are not limited in this context.

Further still, unless expressly stated to the contrary, “or” refers to an inclusive or and not to an exclusive or. For example, “A, B or C” refers to any combination or subset of A, B, C such as (1) A alone, (2) B alone, (3) C alone, (4) A with B, (5) A with C, (6) B with C, and (7) A with B and with C. As used herein, the phrase “at least one of A and B” is intended to refer to any combination or subset of A and B such as (1) at least one A, (2) at least one B, and (3) at least one A and at least one B. Similarly, the phrase “at least one of A or B” is intended to refer to any combination or subset of A and B such as (1) at least one A, (2) at least one B, and (3) at least one A and at least one B.

Moreover, in the foregoing specification, specific embodiments have been described. However, one of ordinary skill in the art appreciates that various modifications and changes can be made in view of aspects of this disclosure without departing from the scope of the invention as set forth in the claims below. Accordingly, the specification and figures are to be regarded in an illustrative rather than a restrictive sense, and all such modifications made in view of aspects of this disclosure are intended to be included within the scope of present teachings.

Additionally, the benefits, advantages, solutions to problems, and any element(s) that may cause any benefit, advantage, or solution to occur or become more pronounced are not to be construed as a critical, required, or essential features or elements of any or all the claims.

Furthermore, although certain example methods, apparatus and articles of manufacture have been disclosed herein, the scope of coverage of this patent is not limited thereto. On the contrary, this patent covers all methods, apparatus and articles of manufacture fairly falling within the scope of the claims of this patent.

1. A system for detecting changes in esophageal tissue properties, the system comprising:
a probe configured for positioning in a patient's esophagus, the probe including one or more tendrils that can be manipulated be a user within the esophagus, wherein the one or more tendrils are configured to contact respective esophageal tissue at different locations in the esophagus, wherein each tendril includes a plurality of nodes spaced apart along the tendril, and wherein each node of the plurality of nodes is configured to be used to introduce signals into esophageal tissue and/or receive signals affected by esophageal tissue; and
a data analysis unit configured to:
introduce a first signal into esophageal tissue using a first node;
determine a value of a characteristic indicative of changes to esophageal tissue at the first node or between a second node and a third node on the same tendril or different tendrils; and
provide an indication of changing tissue properties from a determined baseline value.

2. The system of claim 1, wherein determining the value of the characteristic based on the second signal received using the second node is also based on a third signal received using the third node.

3. The system of claim 1, wherein the first, second and third nodes are of a first tendril of the one or more tendrils.

4. The system of claim 1, wherein the third node is the first node.

5. The system of claim 1, wherein the first node is of a first tendril of the one or more tendrils, and the second node is of a second tendril of the one or more tendrils.

6. The system of claim 1, wherein the data analysis unit is configured to provide the indication of actual esophageal tissue changes when at least one of the determined values, or a change in the at least one of the determined values, passes a threshold.

7. The system of claim 1, further comprising an optical fiber having a plurality of temperature sensors, wherein the data analysis unit is further configured to:
determine a plurality of temperature values of respective esophageal tissue at respective ones of the plurality of temperature sensors; and
provide an indication of potential esophageal tissue changes based on at least one of the determined temperature values.

8. The system of claim 7, wherein the temperature sensors comprise fiber Bragg gratings spaced apart along the optical fiber, with each of the fiber Bragg gratings being configured to reflect a wavelength of light.

9. The system of claim 8, wherein the data analysis unit comprises:
a light source configured to emit light into the optical fiber; and
a wavelength interrogator configured to determine the wavelengths of light reflected by the plurality of fiber Bragg gratings.

10. The system of claim 7, wherein the temperature sensors utilize Rayleigh scatter to measure temperature along the length of the optical fiber.

11. The system of claim 7, wherein the temperature sensors comprise thermocouples spaced apart along the length of the tendril.

12. The system of claim 7, wherein a first tendril of the one or more tendrils includes the optical fiber.

13. The system of claim 7, wherein the data analysis unit is configured to provide the indication of potential esophageal tissue changes when at least one of the determined temperature values exceeds a threshold.

14. The system of claim 1, wherein the data analysis unit is configured to determine the value of the characteristic of esophageal tissue between the second and third nodes based upon one or more differences between the first and second signals.

15. The system of claim 1, wherein the data analysis unit is configured to determine the value of the characteristic of esophageal tissue between the second and third nodes based upon one or more differences between the second signal and a third signal received using the third node.

16. The system of claim 15, wherein the first signal is a current-controlled signal, the one or more differences include a difference in voltage, and the characteristic is at least one of a bioimpedance, a resistance, a reactance, or a conductance.

17. The system of claim 15, wherein the first signal is a sound wave, the one or more differences includes a propagation time, and the characteristic is a shear-wave velocity.

18. The system of claim 17, wherein the sound wave is an ultrasonic wave.

19. The system of claim 1, wherein the probe further includes a photoplethysmography (PPG) sensor.

20. The system of claim 1, further comprising an optical fiber having a plurality of sensors, wherein the data analysis unit is further configured to:
determine a plurality of strain values at respective ones of the plurality of sensors; and
estimate a location of the optical fiber in the esophagus based on the strain values.

21. The system of claim 20, wherein the sensors comprise fiber Bragg gratings or Rayleigh scattering-type strain sensors.

22. The system of claim 1, further comprising electromagnetic sensors configured to be used to triangulate the position of the tendrils in space.

23. The system of claim 1, wherein a first tendril of the one or more tendrils includes a retraction mechanism configured to move the esophagus away from a cardiac ablation site.

24. The system of claim 23, wherein the retraction mechanism includes an air cavity that, when pressurized in the esophagus, moves the esophagus.

25. The system of claim 23, wherein the retraction mechanism is configured to induce a vacuum in the space between the esophageal wall and the probe to move the tissue away from the heart.

26. The system of claim 1, wherein a first tendril of the one or more tendrils is pre-strained, and includes an inner core configured to be selectively inserted into or withdrawn from the first tendril by the user to move the esophagus.

27. The system of claim 26, further including an actuator wire adjacent to the core to induce a bending moment on the core.

28. The system of claim 1, further comprising:
a signal processor configured to analyze received signals; and
an analog multiplexer to communicate the second signal to the signal processor.

29. The system of claim 1, wherein the indication is one or both of a visual warning and an audio warning.

30. A method for detecting changes in esophageal tissue properties due to cardiac ablation, the method comprising:
deploying a probe in a patient's esophagus, wherein the probe includes a sheath and a plurality of tendrils extendible by a user from an end of the sheath, wherein the plurality of tendrils are configured to contact respective esophageal tissue at different locations in the esophagus when extended from the end of the sheath, wherein each tendril of the plurality of tendrils includes a plurality of nodes spaced apart along the tendril, and wherein each node of the plurality of nodes is configured to introduce signals into esophageal tissue and/or receive signals affected by esophageal tissue;
introducing a first signal into esophageal tissue using a first node;
determining, during a cardiac ablation procedure and based on a second signal received using a second node, a value of a characteristic indicative of actual changes to esophageal tissue properties of the patient's esophagus in proximity to a cardiac ablation site and between the second node and a third node;
determining, during the cardiac ablation procedure and based on the value, whether actual esophageal tissue change has occurred; and
providing, during the cardiac ablation procedure, an indication of actual esophageal tissue change when determining that actual esophageal tissue change has occurred.

31.-44. (canceled)

45. A non-transitory, machine-readable storage medium comprising instructions that, when executed, cause a system for detecting actual esophageal tissue changes due to cardiac ablation to, after a probe is deployed in a patient's esophagus, wherein the probe includes a sheath and a plurality of tendrils extendible by a user from an end of the sheath, wherein the plurality of tendrils are configured to contact respective esophageal tissue at different locations in the esophagus when extended from the end of the sheath, wherein each tendril of the plurality of tendrils includes a plurality of nodes spaced apart along the tendril, and wherein each node of the plurality of nodes is configured to introduce signals into esophageal tissue and/or receive signals affected by esophageal tissue:
introduce a first signal into esophageal tissue using a first node;
determine, during a cardiac ablation procedure and based on a second signal received using a second node, a value of a characteristic indicative of actual change to esophageal tissue of the patient's esophagus in proximity to a cardiac ablation site and between the second node and a third node;
determine, during the cardiac ablation procedure and based on the value, whether actual esophageal tissue change has occurred; and
provide, during the cardiac ablation procedure, an indication of actual esophageal tissue change when determining that actual esophageal tissue change has occurred.

46.-52. (canceled)
